(19) United States
(12) Patent Application Publication
(10) Pub. No.: US 2026/0061217 A1
(43) Pub. Date: Mar. 5, 2026

(54) LIGHT THERAPY DEVICE

(71) Applicant: SHENZHEN KAIYAN MEDICAL EQUIPMENT CO., LTD, Shenzhen (CN)

(72) Inventors: ALAIN DIJKSTRA, Amstelveen (NL); Zeng Xianqi, Shenzhen (CN)

(73) Assignee: SHENZHEN KAIYAN MEDICAL EQUIPMENT CO., LTD, Shenzhen (CN)

(21) Appl. No.: 19/365,248

(22) Filed: Oct. 22, 2025

Related U.S. Application Data
(63) Continuation-in-part of application No. 18/679,503, filed on May 31, 2024.

(51) Int. Cl. A61N 5/06; A61N 1/04; A61N 1/32

(52) U.S. Cl. CPC A61N 5/0616; A61N 1/0468; A61N 1/0492; A61N 1/328; A61N 2005/0628; A61N 2005/0632; A61N 2005/0645; A61N 2005/0651; A61N 2005/0659; A61N 2005/0663

(57) ABSTRACT

A light therapy device comprises a flexible casing made with flexible silicone material. A Flexible Printed Circuit Board (FPCB) is installed over a front portion of the flexible casing. The FPCB comprises a plurality of stimulation elements and at least one pair of primary electrodes for therapeutic and recreational purpose. A detachable controller is attached over a back portion of the flexible casing. The controller comprises at least one pair of secondary electrodes, a rechargeable battery, and a user interface. The at least one pair of secondary electrodes engages with the at least one pair of primary electrodes to transmit the power from the rechargeable battery to the FPCB. Further, a flexible diaphanous sheet mounted over the FPCB through which the light therapy is provided to a user.

### CROSS-REFERENCE TO RELATED APPLICATION

This application is a continuation-in-part of U.S. patent application Ser. No. 18/679,503, filed May 31, 2024, entitled “Light Therapy Device”, the entire contents of which are incorporated herein by reference.

### FIELD OF THE INVENTION

The present invention generally relates to the domain of therapeutic and recreational devices. More specifically, the present invention relates to devices that can stick to an individual's skin to provide various kinds of stimulation, including irradiation, heating, cooling, and vibrations.

### BACKGROUND OF THE INVENTION

Various therapeutic devices/patches have been developed for therapeutic and recreational purposes for quite some time. These devices utilize electrodes, heating elements, cooling mechanisms, and irradiation sources like LEDs and lasers to deliver energy to specific body areas. While commonly employed for pain relief, specific therapy devices serve the purpose of facilitating wound healing. Traditional bandages or patches are conventionally designed for single use, thereby exacerbating the issue of medical waste. Furthermore, conventional patches frequently induce discomfort when detached from the user's body. Furthermore, traditional patches, when adhered to the user's skin, may fail to deliver effective therapy due to the uneven shape of the skin, such as in the area below the eyes. Addressing these described challenges, the present invention introduces an innovative approach to light therapy devices to minimize medical waste and enhance user comfort during use and removal.

US20160015962A1 discloses a flexible patch that emits light in the UV, visible, and/or infrared electromagnetic spectrums. The patch includes a feedback process and system using a plurality of sensors and a controller on the patch to accelerate the wound healing process by providing adaptable, controlled light exposure and electrical stimulation. The patch also monitors the healing process for signs of infection and eliminates bacterial infections by sanitizing the infected site and transforming the information through wireless mode to a central location for storage and interpretation by a physician. The disclosed patch also receives feedback from the physician and provides the same to the user. In addition, the physician from the remote location can also instruct the user on how to operate.

The prior art, as exemplified by U.S. Pat. No. '962 reveals a flexible therapy patch designed for administering therapeutic sessions to aid in the healing of wounds on a user's body. The disclosed patch in the above prior art facilitates communication between the user and a physician by exchanging information and provides continuous monitoring of the healing process while eliminating bacteria or fungi from the wound. Despite these commendable features, a notable limitation of the prior art is its non-reusability. Moreover, the uneven shape of certain body areas, such as the region below the eye, poses a challenge during patch usage, as the adhesive tape alone may not suffice to cover the entire irregular surface. Building upon this prior art, the present invention introduces improvements to address these shortcomings, including reusability and enhanced adaptability to uneven body contours.

US20220226668A1 discloses an LED patch for skin care. The LED patch includes a substrate part, a circuit pattern part formed in at least two layers on the substrate part, a light source part including one or more LEDs mounted on one surface of the substrate part, and a cover part configured to cover at least one surface of the substrate part. The disclosed LED patch is utilized to provide a therapeutic session to a user as per the condition/desire of the user.

The above prior art, US'668, disclosed an LED patch helpful in skin care. The LED patch includes a cover part, which makes the LED patch moisture-free. The cover part is made of silicon material, providing comfort to the user using the LED patch. However, the re-usage of the LED patch is not disclosed. Also, the design of the disclosed LED patch is complicated.

Therefore, there is a need in the art for light therapy-based devices that do not suffer from the aforementioned deficiencies.

Existing light and microcurrent therapy devices having in-built controllers provide compactness and ease of use, yet may limit interchangeability, hygiene, and battery serviceability.

In some cases, users prefer a therapy patch in which the control module can be detached for charging, replacement, or use with different patch sizes or electrode configurations.

Furthermore, traditional wearable therapy devices lack adaptive intensity control, such as adjustment based on ambient lighting or skin contact conditions. Therefore, there exists a need for a dual-architecture therapy system offering both an integrated controller configuration and an alternative detachable controller embodiment, each providing enhanced convenience, adaptability, and reusability.

### OBJECTS OF THE INVENTION

Some of the objects of the invention are as follows:

An object of the present invention is to develop a device designed to administer light therapy to a specific body area, aiming to assist in alleviating pain for the user.

Another object of the present invention is to develop a device that minimizes medical waste.

Another object of the present invention is to develop a device that ensures the user does not experience discomfort while using the device.

Another object of the present invention is to develop a device that can be used multiple times, promoting reusability.

Another object of the present invention is to develop a device that is rechargeable, enhancing the longevity and sustainability of the device.

Another object of the present invention is to develop a water-resistant device, enhancing the durability and usability of the device in various conditions.

Another object of the present invention is to develop a lightweight device, aiming to provide comfort and convenience to the user during usage.

Yet another object of the present invention is to develop a device that is capable of emitting various lights, including blue light, red light, etc.

### SUMMARY OF THE INVENTION

According to an embodiment of the present invention, there is provided a light therapy device. The proposed device comprises a patch and a detachable controller. Further, the patch comprises a flexible casing made with silicone material. The flexible casing further comprises a front portion, a back portion, and a peripheral wall. The peripheral wall is crafted over the periphery of the front portion. The patch further includes a plurality of protrusions of a predetermined length crafted over the flexible casing. Further, a Flexible Printed Circuit Board (FPCB) installed over the front portion of the flexible casing. The Flexible Printed Circuit Board (FPCB) comprises a plurality of stimulation elements. The patch further includes a flexible diaphanous sheet made with liquid silicone material and is arranged over the Flexible Printed Circuit Board (FPCB). Moreover, a plurality of cuts crafted over the periphery of the Flexible Printed Circuit Board (FPCB) at a predetermined distance between each of the cuts. The number of cuts is equivalent to the number of protrusions. Further, each of the cuts is engaged with a corresponding protrusion. Furthermore, at least one deformable element is inbuilt within the flexible casing.

In one embodiment of the invention, at least one pair of through-holes is formed over the back portion of the flexible casing.

In one embodiment of the invention, the flexible casing is constructed from opaque material to obstruct the passage of light through the flexible casing.

In one embodiment of the invention, the Flexible Printed Circuit Board (FPCB) comprises at least one pair of primary electrodes passing through the at least one pair of through-holes.

In one embodiment of the invention, the detachable controller is attached to the back portion of the flexible casing. The detachable controller comprises a hollow housing that includes at least one pair of secondary electrodes, the at least one pair of secondary electrodes magnetically attached to the at least one pair of primary electrodes. Further, a rechargeable battery is installed within the controller to power the light therapy device. Moreover, a user interface is crafted over the hollow housing and is electrically connected to the rechargeable battery to operate the light therapy device.

In one embodiment of the invention, the user interface includes but is not limited to a push button and a touch panel.

In one embodiment of the invention, the shape of the patch includes but is not limited to a star shape, a moon shape, a hexagon shape, a heptagon shape, an octagon shape, and a heart shape.

In one embodiment of the invention, the plurality of stimulation elements is selected from a group consisting of irradiation sources, heating elements, cooling elements, vibration elements, ultrasonic wave generators, electrodes, and combinations thereof.

In one embodiment of the invention, the irradiation sources are configured to emit electromagnetic radiation in a wavelength range of 300 nm to 1200 nm.

In one embodiment of the invention, the light therapy device is detachably attached to a user's skin using an adhesive agent to enable a user to reuse the light therapy device multiple times.

In one embodiment of the invention, the at least one deformable element is made with a metallic material.

In one embodiment of the invention, an insulation separates the Flexible Printed Circuit Board (FPCB) and the primary electrodes from the deformable element.

In one embodiment of the invention, the adhesive agent includes but is not limited to a glue, a gel, and a double-sided tape.

In one embodiment of the invention, the flexible diaphanous sheet is sealed with the Flexible Printed Circuit Board (FPCB) via an encapsulating glue.

In one embodiment of the invention, the flexible diaphanous sheet is skin-friendly. In one embodiment of the invention, the light therapy device is water resistant.

In one aspect, the system includes a therapy patch having an in-built controller enclosed within the patch housing. The controller includes a rechargeable power source, a control circuit, and driver electronics for a plurality of light-emitting diodes or microcurrent electrodes. A charging case or alternate storage case is provided for wirelessly charging or safely storing the patch when not in use. The integrated configuration offers compactness and convenient portability.

In another aspect, the system includes a therapy patch configured to engage with a detachable controller module. The detachable controller comprises a hollow housing containing a control board, adapter board, and rechargeable battery arranged on opposite sides of the battery for compact assembly. Secondary magnetic or conductive electrodes on the controller are configured to engage corresponding primary electrodes on the patch to form a detachable electrical and mechanical connection.

The controller further includes a user interface for selecting therapy modes, and may incorporate an ambient light sensor to automatically adjust therapy intensity. A charging and storage case may be used to recharge and protect the detachable controller.

In the context of the specification, the term “flexible substrate” refers to a base material that supports the placement and interconnection of electrical components, while allowing the overall structure to bend or conform to non-planar surfaces. Examples include polyimide film, thermoplastic elastomers, and silicone-based sheets.

In the context of the specification, the term “LED module” refers to one or more light-emitting diode (LED) elements that are electrically connected and configured to emit light of specific wavelengths suitable for therapeutic purposes. The LED module may include drive circuitry, heat dissipation structures, and optical elements such as lenses or diffusers to control light distribution.

Unless otherwise stated, the term “light” as used in this specification encompasses electromagnetic radiation in the visible (380-780 nm) and infrared (780 nm-1000 nm) ranges, particularly red light (620-750 nm) and near-infrared (750-1400 nm) wavelengths commonly used in photobiomodulation therapy. Particular wavelengths which may be selected as the dominant emissive wavelength may include the follow, without any preference to be indicated by order: 400 nm, 405 nm, 420 nm, 430 nm, 450 nm, 465 nm, 515 nm, 530 nm, 532 nm, 590 nm, 630 nm, 633 nm, 640 nm, 650 nm, 655 nm, 660 nm, 670 nm, 680 nm, 780 nm, 785 nm, 810 nm, 830 nm, 840 nm, 850 nm, 860 nm, 870 nm, 904 nm, 915 nm, 980 nm, 1015 nm, 1060 nm, 1065 nm, 1070 nm, 1200, and 1400 nm. As used herein, the term “light therapy” refers to the use of one or more light sources of any type that emit light with a wavelength between about 400 and 1400 nm. The device may also emit blue or ultraviolet light for surface-level treatments such as acne reduction or microbial control.

The red light (approximately 630-660 nm) penetrates deeply into the scalp to stimulate blood circulation and enhance hair follicle activity, thus promoting hair growth and repair. Blue light (around 415-470 nm) exhibits antibacterial properties and is effective in treating scalp acne and reducing inflammation. Green light (approximately 520-540 nm) can help reduce pigmentation and soothe sensitive or irritated scalp tissue. Yellow light (around 580-600 nm) improves oxygen exchange in the cells and aids in detoxifying the scalp, while near-infrared light (800-850 nm) reaches deeper layers to accelerate healing and reduce pain.

In the context of the specification, the term “light source” or “phototherapy source” etc. refers to a source emitting coherent laser light, or light-emitting diodes (“LEDs”). The term “light therapy” refers to light generated from any of the sources, such as lasers, LED sources, Super luminous diodes (“SLD”), or Organic light-emitting diodes (OLED).

In the context of the specification, the term “stimulation element” refers to may include, but is not limited to, phototherapy, micro-current, magneto therapy, cooling, heating, vibration, electrical pulses, or other forms of therapeutic output.

In the context of the specification, the term “electrotherapy,” “electrical stimulation,” or “microcurrent therapy” refers to the application of therapeutic current to the human body, regardless of the precise frequency, waveform, or current intensity.

In the context of the specification, the term “electrotherapy” may refer to different types of electrotherapy, including but not limited to galvanic, electrotherapy, iontophoresis, microcurrent, and EMS (Electrical Muscle Stimulation), TENS (Transcutaneous Electrical Nerve Stimulation).

### BRIEF DESCRIPTION OF DRAWINGS

The accompanying drawings illustrate the best mode for carrying out the invention as presently contemplated and set forth hereinafter. The present invention may be more clearly understood from a consideration of the following detailed description of the preferred embodiments taken in conjunction with the accompanying drawings wherein like reference letters and numerals indicate the corresponding parts in various figures in the accompanying drawings, and in which:

FIG. 1A illustrates a perspective view of a light therapy device in which a detachable controller is detached from a patch, in accordance with an embodiment of the present invention;

FIG. 1B illustrates a perspective view of the light therapy device in which the detachable controller is assembled with the patch, in accordance with an embodiment of the present invention;

FIG. 2A illustrates an exploded view of the patch, in accordance with an embodiment of the present invention;

FIG. 2B illustrates an exploded view of a star shape patch of FIG. 2A.

FIG. 3 illustrates an exploded view of the detachable controller, in accordance with an embodiment of the present invention.

FIG. 4A illustrates an exploded view of the patch designed with a deformable element, in accordance with an embodiment of the present invention.

FIG. 4B illustrates an exploded view of the patch designed with the deformable element in another configuration, in accordance with an embodiment of the present invention.

FIG. 5A illustrates a bottom view of the light therapy device in various shapes, in accordance with an embodiment of the present invention; and

FIG. 5B illustrates a top view of the light therapy device in various shapes of FIG. 5A, in accordance with an embodiment of the present invention.

FIG. 6 illustrates a top view of another configuration of a light therapy device, in accordance with an embodiment of the present invention.

FIG. 7 illustrates an exploded view of the light therapy device, in accordance with an embodiment of the present invention.

FIG. 8 illustrates an exploded view of another configuration of a light therapy device, in accordance with an embodiment of the present invention.

FIG. 9 illustrates an exploded view of a charging device, in accordance with an embodiment of the present invention.

FIG. 10 illustrates a top view of a charging device showing the open position of an outer cover, in accordance with an embodiment of the present invention.

FIG. 11 illustrates a perspective view of another configuration of a light therapy patch and a corresponding controller, in accordance with an embodiment of the present invention.

FIG. 12 illustrates a bottom view of the light therapy patch, in accordance with an embodiment of the present invention.

FIG. 13 illustrates a top view of the controller, in accordance with an embodiment of the present invention.

FIG. 14 illustrates a top exploded view of another configuration of a light therapy device, in accordance with an embodiment of the present invention.

FIG. 15 illustrates a bottom exploded view of the light therapy device, in accordance with an embodiment of the present invention.

FIG. 16 illustrates a top view of yet another configuration of a light therapy device, in accordance with an embodiment of the present invention.

FIG. 17 illustrates a cross-sectional view of the light therapy device, in accordance with an embodiment of the present invention.

FIG. 18 illustrates a perspective view of another configuration of a light therapy patch and a controller, in accordance with an embodiment of the present invention.

FIG. 19 illustrates an exploded view of the controller, in accordance with an embodiment of the present invention.

### DETAILED DESCRIPTION OF THE INVENTION

Embodiments of the present invention disclosure will be described more fully hereinafter with reference to the accompanying drawings in which like numerals represent like elements throughout the figures, and in which example embodiments are shown.

The detailed description and the accompanying drawings illustrate the specific exemplary embodiments by which the disclosure may be practiced. These embodiments are described in detail to enable those skilled in the art to practice the invention illustrated in the disclosure. It is to be understood that other embodiments may be utilized, and other changes may be made, without departing from the spirit or scope of the present disclosure. The following detailed description is, therefore, not to be taken in a limiting sense, and the scope of the present invention disclosure is defined by the appended claims. Embodiments of the claims may, however, be embodied in many different forms and should not be construed as limited to the embodiments set forth herein.

In the context of this specification, terms like “light” and “illumination”, etc. refer to electromagnetic radiation in wavelength ranges varying from the visible light wavelengths (380-750 nm) to Infrared (IR) wavelengths (750 nm-1400 nm), wherein the range is inclusive of visible light and IR wavelengths. The IR radiation may also be categorized into several categories according to respective wavelength ranges, which are again envisaged to be within the scope of this invention. A commonly used subdivision scheme for IR radiation includes Near IR (0.75-1.4 μm), Short-Wavelength IR (1.4-3 μm), Mid-Wavelength IR (3-8 μm), Long-Wavelength IR (8-15 μm) and Far IR (15-1000 μm).

In the context of the specification, “Light Emitting Diodes (LEDs)” are envisaged to be semiconductor devices that emit electromagnetic radiation when current is applied through them. LEDs are characterized by their superior power efficiencies, smaller sizes, rapidity in switching, physical robustness, and longevity when compared with incandescent or fluorescent lamps. In that regard, the plurality of LEDs may be through-hole type LEDs (generally used to produce electromagnetic radiations of red, green, yellow, blue, and white colors), Surface Mount LEDs, Bi-color LEDs, Pulse Width Modulated RGB (Red-Green-Blue) LEDs, and high-power LEDs, etc.

Materials used in the construction of LEDs may vary from one embodiment to another depending upon the frequency of radiation required. Different frequencies can be obtained from LEDs made from pure or doped semiconductor materials. Commonly used semiconductor materials include nitrides of Silicon, Gallium, Aluminum, Boron, Zinc, Selenide, etc., in pure form or doped with elements such as Aluminum and Indium, etc. For example, red and amber colors are produced from Aluminum Indium Gallium Phosphide (AlGaInP) based compositions, while blue, green, and cyan use Indium Gallium Nitride based compositions. White light may be produced by mixing red, green, and blue lights in equal proportions, while varying proportions may be used to generate a wider color gamut. White and other colored lightings may also be produced using phosphor coatings such as Yttrium Aluminum Garnet (YAG) in combination with a blue LED to generate white light and Magnesium doped potassium fluorosilicate in combination with a blue LED to generate red light. Additionally, near Ultraviolet (UV) LEDs may be combined with europium-based phosphors to generate red and blue lights and copper and zinc-doped zinc sulfide-based phosphors to generate green light.

In addition to conventional mineral-based LEDs, one or more LEDs may also be provided on an organic LED (OLED) flexible panel or an inorganic LED-based flexible panel. Such OLED panels may be generated by depositing organic semiconducting materials over Thin Film Transistor (TFT) based substrates. Further, discussion on generation of OLED panels can be found in Bardsley, J. N (2004), “International OLED Technology Roadmap”, IEEE Journal of Selected Topics in Quantum Electronics, Vol. 10, No. 1, that is included herein in its entirety, by reference. An exemplary description of flexible inorganic light-emitting diode strips can be found in granted U.S. Pat. No. 7,476,557 B2, titled “Roll-to-roll fabricated light sheet and encapsulated semiconductor circuit devices”, which is included herein in its entirety, by reference.

In several embodiments, the one or more LEDs may also be micro-LEDs described through U.S. Pat. Nos. 8,809,126 B2, 8,846,457 B2, 8,852,467 B2, 8,415,879 B2, 8,877,101 B2, 9,018,833 B2 and their respective family members, assigned to NthDegree Technologies Worldwide Inc., which are included herein by reference, in their entirety. The one or more LEDs, in that regard, may be provided as a printable composition of the micro-LEDs, printed on a substrate.

In the context of the specification, the phrase “diaphanous material” refers to a material that allows at least a portion of one or more forms of electromagnetic radiation (such as Infrared, Ultraviolet, X-rays, Visible Light, Microwaves, Radio Waves, etc.) to pass through them. The diaphanous materials can be transparent (allowing one or more forms of electromagnetic radiation to pass through with minimal scattering) or translucent (allowing one or more forms of electromagnetic radiation to pass through with appreciable diffusion or scattering). Diaphanous materials can be dense, like glass, or have an open structure, like wire mesh or a woven fabric.

The proposed therapeutic light therapy device is designed to administer a healing session to the user. The light therapy device comprises a patch and a detachable controller. The detachable controller includes a hollow housing, a user interface, and a rechargeable battery. In addition, at least one pair of secondary electrodes is crafted over a bottom portion of the hollow housing of the detachable controller.

Further, the patch is constructed with a flexible casing made of silicone material and is available in various shapes, such as stars, moons, hexagons, heptagons, octagons, and hearts. The flexible casing comprises a front portion, a back portion, and a peripheral wall. The front portion is surrounded by a Flexible Printed Circuit Board (FPCB) that comprises multiple stimulation elements. The stimulation elements include but are not limited to a plurality of Light Emitting Diodes (LEDs), a plurality of lasers, a plurality of tensed electrotherapy elements, and a plurality of heating elements. The stimulation elements are responsible for emitting diverse colors of light/heat/electrotherapy to provide therapy to the user. Further, the Flexible Printed Circuit Board (FPCB) also includes at least one pair of primary electrodes, which are magnetically connected to the at least one pair of secondary electrodes to transmit the power from the rechargeable battery to the Flexible Printed Circuit Board (FPCB).

In addition, the periphery of the Flexible Printed Circuit Board (FPCB) is designed with a plurality of cuts. On the other hand, a plurality of protrusions protruded on the flexible casing. The number of the plurality of cuts is equivalent to the number of the plurality of protrusions. Each of the cuts engages with each of the corresponding protrusions to hold the FPCB strongly and securely within the patch. In addition, a flexible diaphanous sheet lay over the Flexible Printed Circuit Board (FPCB). The flexible diaphanous sheet is made of a liquid silicone material. While manufacturing, there are chances that the liquid silicone may enter between the Flexible Printed Circuit Board (FPCB) and the flexible casing. The engagement of the cuts with the protrusions restricts the liquid silicone from entering between the FPCB and the flexible casing. Further, the flexible diaphanous sheet herein allows the emitted light to pass through the flexible diaphanous sheet and deliver therapeutic benefits to the user.

Further, to ensure a secure grip between the user's skin and the light therapy device, a deformable element, which may be a metallic wire or sheet or grid of metallic wire/sheet, is integrated into the flexible casing. Furthermore, an adhesive agent is detachably attached to the patch to affix the light therapy device over the user's body portion for starting the therapeutic session. The adhesive agent for attaching the patch over the user's skin is skin-friendly and makes the device used multiple times. Some of the examples of skin-friendly adhesive agents may include, but are not limited to, silicones polydimethylsiloxane (PDMS) and silicone acrylates, acrylics, hydrocolloids, cyanoacrylates, etc. Further, the adhesive agent can be in the form of a glue, a gel, or double-sided tape. The material of the adhesive agent would be diaphanous.

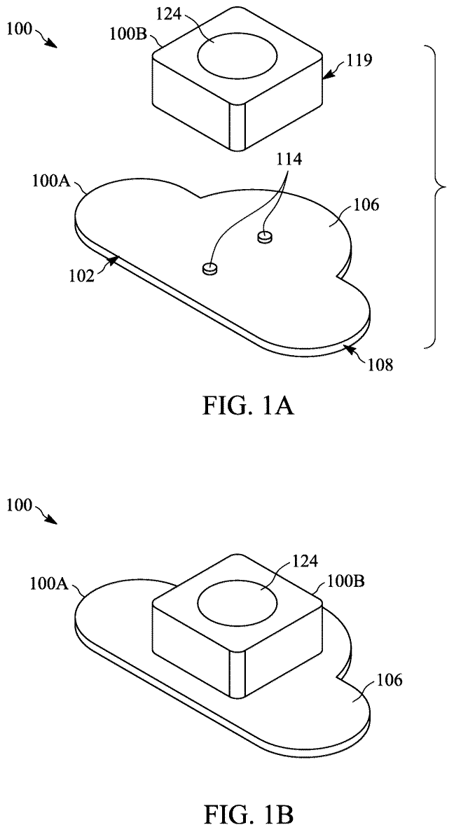

Several embodiments of the present invention will now be elucidated with the help of FIG. 1A-5B.

FIG. 1A illustrates a perspective view of a light therapy device 100 (referred to as “the light therapy device 100”) in which a detachable controller 100B is detached from a patch 100A, in accordance with an embodiment of the present invention. In several embodiments of the present invention, the light therapy device 100 comprises a patch 100A and a detachable controller 100B. The detachable controller 100B is utilized to control the patch 100A through numerous operations, such as switching on/off the light therapy device 100, and changing the lights to provide a therapeutic session to a user.

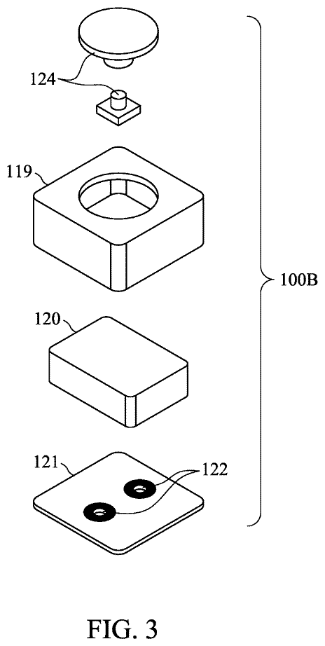

The detachable controller 100B is comprised of a hollow housing 119, a user interface 124, a rechargeable battery (as shown in FIG. 3), and at least one pair of secondary electrodes (as shown in FIG. 3). The user interface 124 may include but is not limited to a push button and a touch panel. The user interface 124 located on the top surface of the detachable controller 100B enabling the user to operate various functions, such as switching the lights or other different modes of operation. Alternatively the user interface 124 can be situated on the side walls of the detachable controller 100B.

In several embodiments of the present invention, the patch 100A comprises a flexible casing 102. The flexible casing 102 is made of a silicone material. The choice of silicone material is deliberate, selected due to the inherent properties such as flexibility, durability, and biocompatibility. The flexibility of the silicone material allows the patch 100A to conform to diverse shapes and contours, ensuring comfort and adaptability to different body surfaces.

In addition, the durable nature of the silicone material contributes to the prolonged effectiveness of the light therapy device 100. Furthermore, the biocompatible properties of silicone, coupled with the electrical insulation capability, mitigate the chances of injuries related to electrical short circuits. Moreover, the flexible casing 102 renders the patch 100A waterproof and resistant to temperature variations.

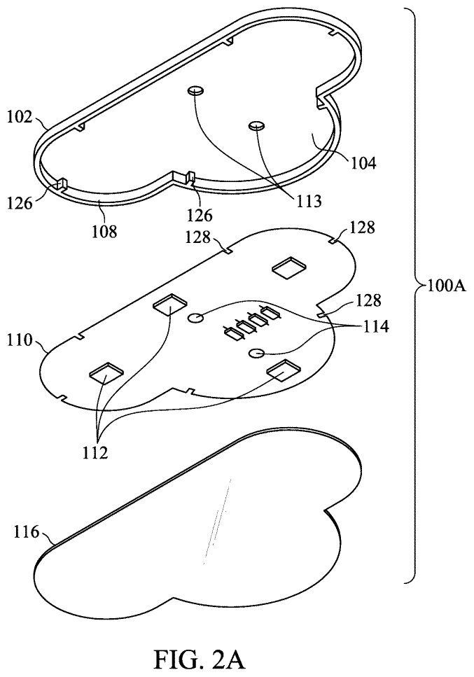

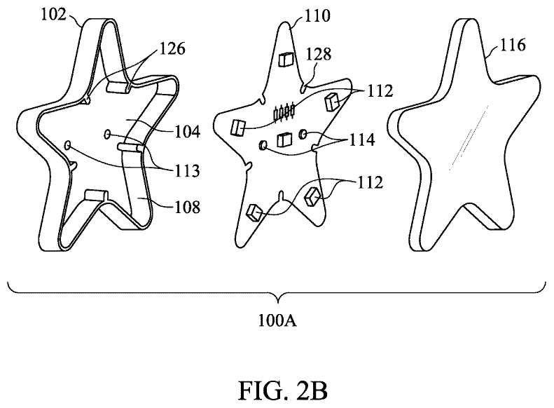

In several embodiments of the present invention, the flexible casing 102 comprises a front portion (104, as shown in FIG. 2A), a back portion 106, and a peripheral wall 108. The peripheral wall 108 is meticulously crafted over the front portion (104, as shown in FIG. 2A). The patch 100A further includes a Flexible Printed Circuit Board (FPCB) (as shown in FIG. 2A), which surrounds the flexible casing 102 from the side of the front portion (104, as shown in FIG. 2A). The Flexible Printed Circuit Board (FPCB) (as shown in FIG. 2A) comprises of a plurality of stimulation elements (as shown in FIG. 2A) and at least one pair of primary electrodes 114. The at least one pair of primary electrodes 114 is utilized to transmit the power from the rechargeable battery (as shown in FIG. 3) to the Flexible Printed Circuit Board (FPCB) (as shown in FIG. 2A).

FIG. 1B illustrates a perspective view of the light therapy device 100, in which the detachable controller 100B is assembled with the patch 100A, in accordance with an embodiment of the present invention. In several embodiments of the present invention, the detachable controller 100B is securely attached to the patch 100A. The at least one pair of primary electrodes 114 is magnetically engaged with the at least one pair of secondary electrodes (as shown in FIG. 3) to transmit the power from the rechargeable battery (as shown in FIG. 3) to the Flexible Printed Circuit Board (FPCB) (as shown in FIG. 2A).

FIG. 2A and FIG. 2B illustrate an exploded view of the patch 100A, and an exploded view of a star-shaped patch 100A, respectively, in accordance with an embodiment of the present invention. In several embodiments of the present invention, the star shape patch 100A has the same configuration as of the patch 100A in FIG. 2A, only the shape of the patch 100A is different.

In several embodiments of the present invention, the patch 100A includes the flexible casing 102, the Flexible Printed Circuit Board (FPCB) 110, and a flexible diaphanous sheet 116 made of a diaphanous material. The diaphanous material may be a Polycarbonate, PMMA or Acrylic, Polyethylene Terephthalate (PET), Amorphous Co-polyester (PETG), Polyvinyl Chloride (PVC), Liquid Silicone Rubber (LSR), Cyclic Olefin Copolymers, Polyethylene (PE), Polystyrene (PS), Thermoplastic polyurethanes (TPU), Polyvinyl butyral (PVB), Co-polymer ethylene vinyl acetate (EVA).

Moreover, the flexible casing 102 is crafted with at least one pair of through-holes 113. The through-holes 113 are utilized for passing the at least one pair of primary electrodes 114 from the flexible casing 102 to magnetically attach the at least one pair of primary electrodes 114 with the at least one pair of secondary electrodes (as shown in FIG. 3).

In several embodiments of the present invention, the peripheral wall 108 is crafted along the periphery of the flexible casing 102, featuring a plurality of protrusions 126 of predetermined length. The plurality of protrusions 126 is strategically oriented towards the center of the flexible casing 102, although it is recognized that alternate configurations may be conceivable by those skilled in the art.

In several embodiments of the present invention, a plurality of cuts 128 is crafted over the periphery of the Flexible Printed Circuit Board (FPCB) 110. The plurality of cuts 128 is engaged with the plurality of the protrusions 126 to make a strong grip in between the Flexible Printed Circuit Board (FPCB) 110 and the flexible casing 102.

In several embodiments of the present invention, the Flexible Printed Circuit Board (FPCB) 110 incorporates the plurality of stimulation elements 112 and the at least one pair of primary electrodes 114. The plurality of stimulation elements 112 may be selected from a group consisting of irradiation sources, heating elements, cooling elements, vibration elements, ultrasonic wave generators, electrodes, and combinations thereof. Furthermore, irradiation sources may be selected from a group consisting of Light Emitting Diodes (LEDs), and lasers. Additionally, the irradiation sources may be configured to operate in one or more of a pulse mode and a continuous mode. In the case of a stimulation element being an irradiation source, the stimulation element would be emitting electromagnetic radiation. The wavelengths of emitted light from the irradiation sources fall within the ranges of 300-1200 nm.

In the case of a stimulation element being a vibration element, the stimulation element would be a vibrating head connected to an eccentric mass rotating motor or a linear resonant motor. In case of a stimulation element being a heating element, the stimulation element may be selected from a group consisting of metal heating elements, ceramic heating elements, semiconductor heating elements, thick film heating elements, polymer-based heating elements, composite heating elements, and combination heating elements. In the case of a stimulation element being a cooling element, the stimulation element may be a thermoelectric cooler, also known as a Peltier heat pump. In the case of a stimulation element being an ultrasonic wave generator, a wave generation head may consist of a quartz crystal fused with a metal plate. The quartz crystal may produce ultrasonic waves due to the piezoelectric effect. Ultrasonic wave therapy may be used in applications such as treatment of chronic pain, improvement in blood circulation, and tissue repair.

In the case of a stimulation element being an electrode, the stimulation element may be embodied as an open-ended conductor. The electrode may then be able to provide Transcutaneous Electrical Nerve Stimulation (TENS), Electronic Muscle Stimulation (EMS), and Microcurrent Electrical Therapy (MET) to the body of a user. TENS therapy uses low-voltage currents to provide pain relief. Electrical impulses are delivered through electrodes placed on the surface of the body of the user. The electrodes are placed at or near nerves where the pain is located or at certain known trigger points. EMS therapy is similar to TENS therapy, the difference being that EMS is applied to key muscle groups instead of a generalized application. The electrical signals in EMS cause certain muscles to undergo contractions and tightening. Moreover, electrical impulses in EMS are stronger when compared with TENS therapy. MET in contrast uses a current of amplitude less than 1 milliampere and a frequency of 0.5 Hz and is indicated for the treatment of pain.

Further, in several embodiments, the flexible diaphanous sheet 116 is mounted over the Flexible Printed Circuit Board (FPCB) 110. The flexible diaphanous sheet 116 is securely attached to the Flexible Printed Circuit Board (FPCB) 110 via encapsulating glue, ensuring a robust connection. The flexible diaphanous sheet 116, made of silicone material, shares properties identical to those of the silicone material of the flexible casing 102. In addition, the silicone material of the flexible diaphanous sheet 116 is translucent. The translucent nature of the sheet facilitates the passage of light without compromising intensity or wavelength effectiveness. Additionally, the silicone material imparts a soft texture to enhance user comfort during the application of the light therapy device 100.

In an alternative embodiment, a set of electrodes, which may be the primary electrodes 114 or another electrode originating from the Flexible Printed Circuit Board (FPCB) 110, may extend from the flexible diaphanous sheet 116 to make direct contact with the user's skin. The tactile engagement of the electrodes with the user's skin initiates the activation of the plurality of tensed electrotherapy elements, thereby administering therapeutic treatment to the user.

FIG. 3 illustrates an exploded view of the detachable controller 100B, in accordance with an embodiment of the present invention. In several embodiments of the present invention, the hollow housing 119 of the detachable controller 100B is of square shape, although it is recognized by those skilled in the art that alternate configurations/shapes may be conceivable. In several embodiments of the present invention, the rechargeable battery 120 incorporated in the detachable controller 100B encompasses various types, including but not limited to nickel-cadmium (NiCd), nickel-metal hydride (NiMH), and lithium-ion (Li-ion). Furthermore, the voltage applied to the rechargeable battery 120 falls within the range of 1.2 volts to 3.7 volts. Further, the user interface 124 enables the user to operate various functions, such as switching the lights or other different modes of operation.

In several embodiments of the present invention, the at least one pair of secondary electrodes 122 is crafted over a bottom portion 121 of the hollow housing 119. The at least one pair of secondary electrodes 122 encompassing both a positive terminal and a negative terminal. The positive terminal and the negative terminal are integral components within the detachable controller 100B and are electrically linked to the rechargeable battery 120.

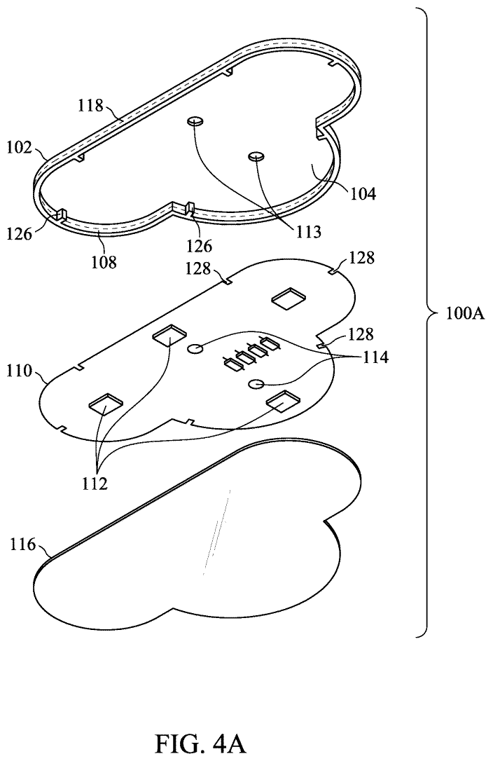

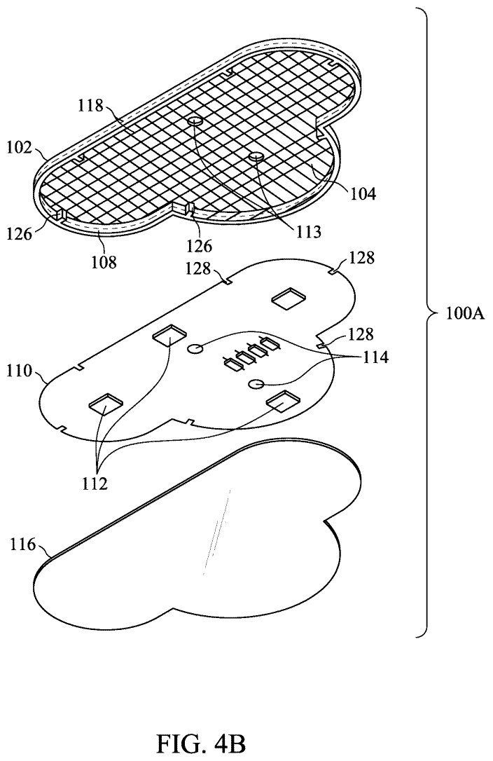

FIG. 4A illustrates an exploded view of the patch 100A designed with a deformable element 118, in accordance with an embodiment of the present invention. In several embodiments of the present invention, the flexible casing 102 of the light therapy device 100 is intrinsically integrated with at least one deformable element 118. The at least one deformable element 118 is preferably crafted from a metallic material comprised of ductile properties, ensuring the deformation of the patch 100A during the application of the patch 100A over the user's body. The choice of a ductile material prevents the at least one deformable element 118 from failing due to deformation. Although, it is recognized that alternate materials may be conceivable for those skilled in the art.

The at least one deformable element 118 in the configuration serves the crucial function of establishing a secure grip between the patch 100A and the user's skin. The secure grip ensures that there is no vacant space left at the periphery of the patch 100A during usage of the patch 100A over the user's body portion. The incorporation of the at least one deformable element 118 enhances the adherence of the light therapy device 100 to the contours of the user's body, optimizing the therapeutic effectiveness.

FIG. 4B illustrates an exploded view of the patch 100A designed with a deformable element 118 in another configuration, in accordance with an embodiment of the present invention. In several embodiments of the present invention, the deformable element 118 may be at the periphery of the flexible casing 102 or, at the back side of the flexible casing 102, or at both the periphery and the back side of the flexible casing 102, (as shown in FIG. 4B). The function of the deformable element 118 is same as described above under the description of FIG. 4A.

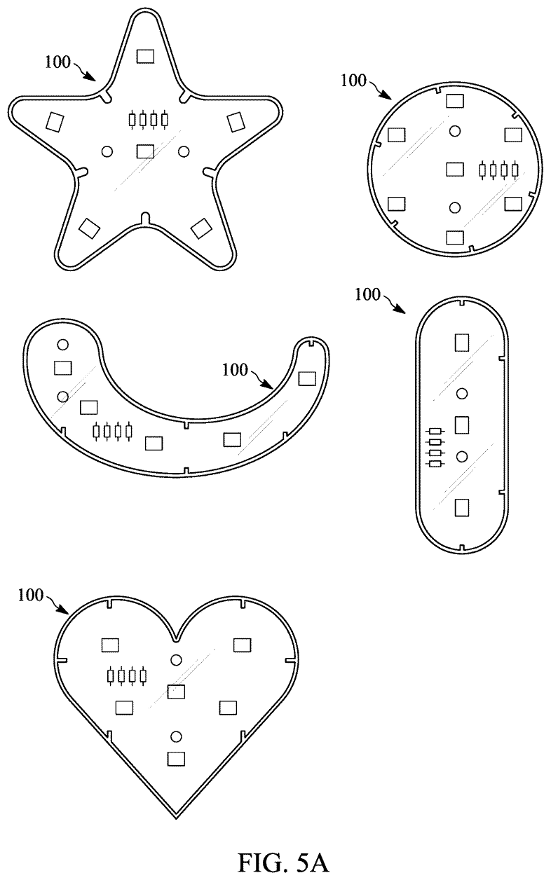

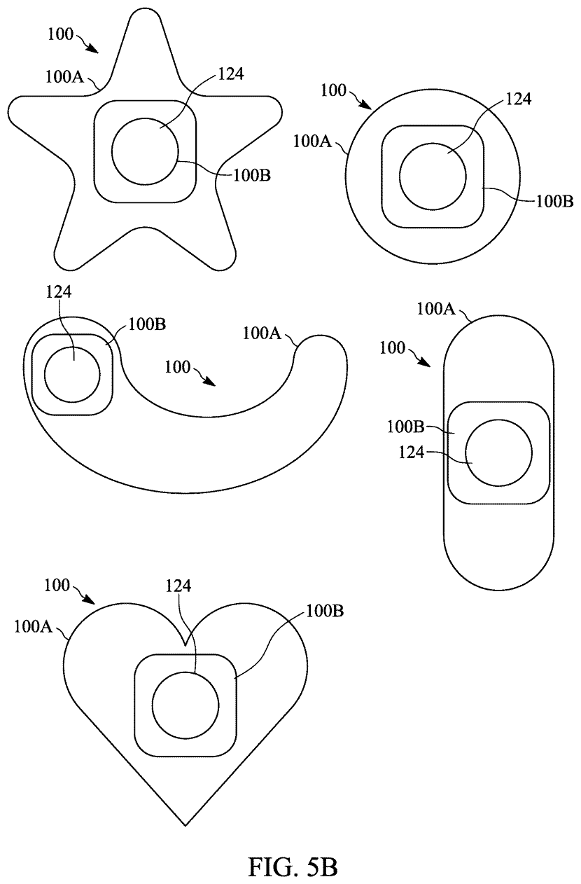

FIG. 5A and FIG. 5B illustrate a bottom view of the light therapy device 100 in various shapes and a top view of the light therapy device 100 in various shapes, respectively, in accordance with an embodiment of the present invention. In numerous embodiments disclosed in the present invention, FIG. 5A and FIG. 5B illustrate a light therapy device 100 featuring various shapes such as circular, moon, and heart, among others, although it is recognized that alternate configurations may be conceivable by those skilled in the art. Despite the diversity in shapes, all iterations of the light therapy device 100 shares a common purpose: to administer therapeutic sessions to users, catering to wound healing and pain relief.

The invention, as described above, offers several advantages. For instance, the light therapy device is very simple in design and construction. Further, the light therapy device uses commonly available materials. The simplicity in design and construction, and the use of commonly available materials allow the light therapy device to be mass-produced with minimal capital expenditure, and to be made available in the market at significantly lower prices. Also, the light therapy device can be reused several times and is, therefore, cost-effective for the end user and minimizes waste generation.

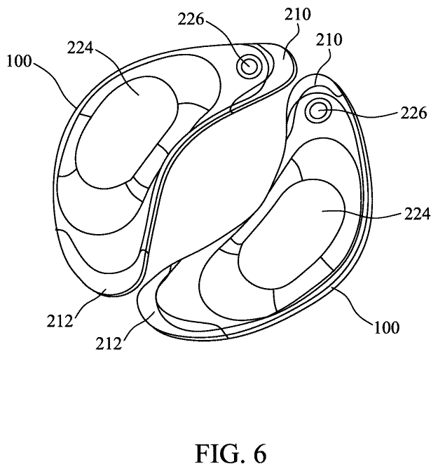

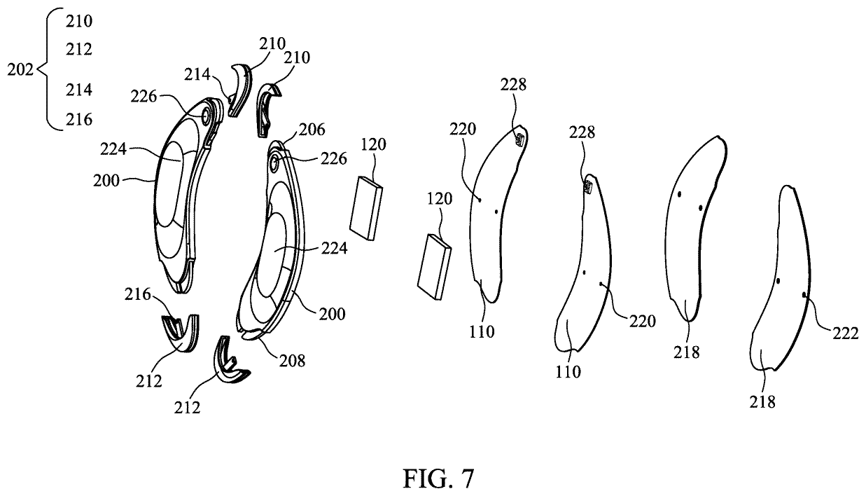

In some embodiments, as shown in FIGS. 6 and 7, the present application provides a light therapy device comprising a main body 200, a circuit board 110, and a microcurrent assembly 202. The main body 200 includes opposing first end 206 and second end 208, within which the circuit board 110 is located. The microcurrent assembly 202 includes a first microcurrent electrode 210 and a second microcurrent electrode 212, each electrically connected to the circuit board 110. The first microcurrent electrode 210 is disposed at the first end 206, while the second microcurrent electrode 212 is disposed at the second end 208, with one functioning as a positive electrode and the other as a negative electrode. When adhered to the skin, the electrodes make contact with the user's body, allowing circuit board 110 to transmit a microcurrent (ranging from 50 μA to 1000 μA, preferably 50 μA to 320 μA) across a broad area of tissue between the two ends (206, 208). This provides wide-ranging stimulation that relieves pain, promotes blood circulation, and reduces inflammation. The extended electrode distance expands the effective treatment area, thus improving therapeutic efficacy and efficiency compared to traditional electrode patches.

In an embodiment, the first end 206 is provided with a first protrusion having a first notch connected to the interior of the main body 200, while one side of the first microcurrent electrode 210 is provided with a first groove that engages with the first protrusion. The microcurrent assembly 202 further includes a first conductive member 214, one end of which is connected to the first groove and the other end of which passes through the first notch to connect with the circuit board 110. The first groove and first protrusion structure allow for precise and rapid positioning of the first microcurrent electrode 210, improving assembly efficiency and preventing misalignment. The first conductive member 214 ensures a stable electrical connection between the electrode and the circuit board 110, while the notch functions as both a passage and a guide, securing the member's position and reducing contact failure risks during use. In one embodiment, the first microcurrent electrode 210 and first conductive member 214 are metallic and integrally injection-molded to enhance assembly simplicity, connection reliability, and structural integrity.

Similarly, the second end 208 includes a second protrusion defining a second notch that connects to the main body's interior. The second microcurrent electrode 212 has a second groove that engages with the second protrusion, and a second conductive member 216 connects between the second groove, the second notch, and the circuit board 110. This mirrored structure ensures secure mechanical positioning and stable electrical connectivity of the second microcurrent electrode 212, thus improving assembly reliability. The second microcurrent electrode 212 and second conductive member 216 may also be metallic and integrally injection-molded to simplify manufacturing and enhance durability.

In an embodiment, a side of the main body 200 has a receiving groove for housing the circuit board 110. The groove's sidewall includes a step, and a patch cover 218 is provided at its notch to press the circuit board 110 against the step, thereby securing it in place. The step may be annular or comprise multiple protrusions to enhance fixation stability. The patch cover 218 may be glued or integrally molded, ensuring effective sealing and protection against moisture or dust. The main body 200 may serve as a backlight component, with the patch cover 218 made of a light-transmitting material. The therapy patch further includes multiple lamp beads electrically connected to the circuit board 110 and disposed adjacent to the patch cover 218. These lamp beads may include light sources of different wavelengths, such as red (580-680 nm±10 nm) and infrared (830-1080 nm), for phototherapy. The red light promotes healing and reduces inflammation, while the infrared light penetrates deeper to relieve muscle and joint pain. Together, these provide combined phototherapy and microcurrent stimulation modes, enhancing therapeutic flexibility. The main body 200 can be formed from opaque flexible materials such as silicone or thermoplastic polyurethane, while the patch cover 218 is preferably made from translucent flexible materials like silicone or polydimethylsiloxane.

In an embodiment, the therapy patch further includes a translucent adhesive on the patch cover 218 to attach the patch to the skin. The adhesive can be a transparent or perforated sticker, transparent tape, or hydrogel layer, providing firm attachment without obstructing light emission. The main body 200, patch cover 218, and circuit board 110 may all be flexible to allow the patch to conform to various body contours. For example, the main body 200 and patch cover 218 can be made of silicone or memory metal-lined silicone, and the circuit board 110 can be a flexible PCB. This flexible configuration ensures comfortable wear and optimal skin contact, improving both microcurrent and light therapy performance. The therapy patch further includes a rechargeable battery 120 housed within the receiving groove and electrically connected to the circuit board 110. A charging electrode assembly having two charging electrodes 220, positive and negative, is electrically connected to the circuit board 110 and exposed through through-holes 222 on the patch cover 218, allowing easy contact-based charging in a matching charging case. The main body 200 portion corresponding to the rechargeable battery 120 forms an outward bulge 224, creating space for a higher-capacity battery (e.g., 60 mAh) to enhance endurance.

In an embodiment, the main body 200 includes an outwardly protruding pressing portion 226 corresponding to a button 228 electrically connected to the circuit board 110 and positioned within a retaining seat. The protruding pressing portion 226 enables the user to control operations such as power and therapy mode switching by pressing the surface, maintaining a seamless, waterproof design. For instance, pressing and holding for two seconds powers the device on or off, and a single short press toggles between light therapy (red+infrared) and combined microcurrent+light therapy modes. A first indicator light connected to the circuit board 110 provides battery and charging status: white for sufficient power, flashing when low, and red when charging interruptions occur. The therapy patch may be crescent-shaped, enabling it to conform to the contours of areas like the face or eyes for targeted therapy. Furthermore, a therapy assembly may include one or more such patches and a charging case configured to recharge and store them. The charging case includes a body and a hinged lid, an internal control board, a second battery (360 mAh), and charging contacts corresponding to the charging electrodes 220 of the patches.

Though the microcurrent electrodes detachably connected to the first end and second end have been described in reference to the light therapy device with an in-built controller, however, in a specific embodiment, the light therapy patch with a detachable controller is also provided with the detachable microcurrent electrodes at the two lateral ends of the phototherapy patch.

In an embodiment, the first microcurrent electrode 210 and second microcurrent electrode 212 may function as a stimulation element capable of delivering diverse therapeutic effects in addition to microcurrent therapy. The electrodes may be configured to operate in different modes such as electrical muscle stimulation (EMS) for promoting muscle activation and toning, heating or cooling therapy for temperature-based relief and relaxation, vibrations and ultrasonic wave therapy for deep tissue stimulation and enhanced metabolic activity. Each mode may be selectively controlled via the circuit board 110 based on preset programs or user selection through the button 228. In EMS mode, low-frequency pulses (e.g., 1-100 Hz) may be generated to induce gentle muscle contractions, while in heating or cooling mode, the electrodes may integrate thermoelectric or resistive elements to deliver controlled warmth or cooling. In ultrasonic mode, the electrodes may function as transducers to emit mechanical vibrations in the ultrasonic range (e.g., 1-3 MHz), promoting microcirculation and enhancing the absorption of active ingredients if used in conjunction with topical agents. Thus, the first microcurrent electrode 210 and the second microcurrent electrode 212 serve as multifunctional therapeutic units capable of providing customizable treatments tailored to various physiological conditions and user needs.

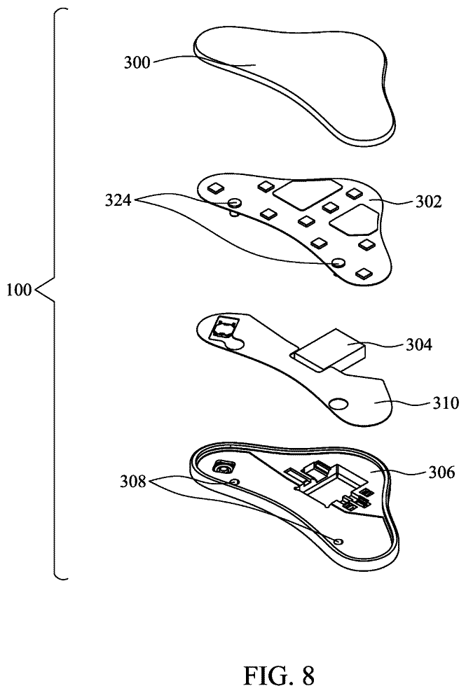

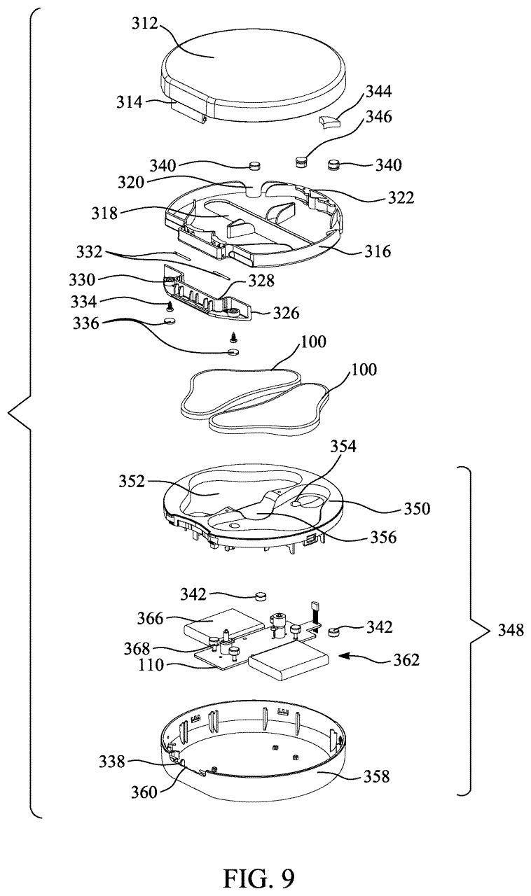

Referring to FIGS. 8 and 9, the present application provides a light therapy charging box capable of conveniently storing, charging, and protecting the light therapy device 100 and its accessories. The light therapy charging box includes a cover and a box body 348, wherein the box body 348 consists of an inner tray 350, a bottom shell 358, and a charging assembly 362. The inner tray 350 is connected to the bottom shell 358, and the charging assembly 362 is mounted between them. The inner tray 350 defines a receiving slot 352 that mates with the back of the light therapy device 100, and a connection terminal 368 is provided at the bottom of the receiving slot 352 for electrical connection to the charging assembly 362. The cover includes an outer cover 312 and an inner cover 316, with the inner cover 316 movably mounted on the outer cover 312. A storage chamber 324 is enclosed between the inner cover 316 and the outer cover 312. The light therapy device 100 can be accommodated within the receiving slot 352 and electrically connected to the charging assembly 362 via the connection terminal 368 for charging. Meanwhile, the storage chamber 324 formed between the inner cover 316 and the outer covers 312 can hold accessories such as stickers, achieving spatial independence between stored items and the light therapy device 100. This prevents interference or damage during storage or transportation. The receiving slot 352 conforms to the back of the light therapy device 100, allowing a snug fit that ensures stability during placement.

In an embodiment, the inner cover 316 includes a first latch 318 designed to facilitate easy opening and closing. The first latch 318 may extend across the length of the inner cover 316, increasing the user's contact area and making the inner cover 316 more accessible. The first latch 318 may also extend through the inner cover 316 as a through-hole, allowing the user to insert fingers directly for effortless opening and closing. This through-hole design also assists in breaking the vacuum between stored stickers and the inner surface of the inner cover 316, simplifying sticker removal. The wall of the storage chamber 324 includes dividers that partition the cavity into two or more storage spaces, allowing organized placement of different accessories. The first latch 318 may extend across both storage spaces, offering a continuous and elongated handle for flexible operation. The dividers may be symmetrically placed on both sides of the first latch 318, thereby enhancing structural stability and preventing deformation of the inner cover 316 due to repeated operation. Each divider may include a first piece and a second piece, with a gap between their opposite ends. This gap increases the spacing between adjacent storage spaces and prevents accessories from obstructing the first latch 318. The dividers can be integrally molded with the inner cover 316 for higher positional accuracy and bonding strength, or fixed to the sidewall of the outer cover 312.

In an embodiment, the inner cover 316 is rotatably connected to the outer cover 312 through a connecting base 326 and a first rotating shaft 332. The connecting base 326 is fixed to the outer cover 312, while the first rotating shaft 332 passes through the connecting base 326 and the inner cover 316, enabling smooth and durable rotation. The connecting base 326 includes a first rotation groove 328, in which one end of the inner cover 316 is embedded and through which the first rotating shaft 332 passes. This groove conceals the rotating shaft and provides guided rotational motion, ensuring stable operation without exposing moving components. The connecting base 326 is detachably fixed to the outer cover 312 through a fastener 334, which is housed within a connecting sleeve 330. The connecting sleeve 330 guides and limits the fastener 334 while preventing exposure, thereby improving appearance and safety. A protective plug 336 can be removably inserted into the connecting sleeve 330 to conceal the fastener 334 and further enhance the aesthetic and safety aspects of the cover. The cover and box body 348 can be connected by various means, such as magnetic attachment or rotational hinges. In one preferred embodiment, the bottom shell 358 includes a second rotating shaft 338 engaging a rotational notch 360 and a rotational connecting piece 314 on the outer cover 312. This structure ensures smooth rotation, prevents accidental detachment, and conceals the shaft for user comfort.

In addition, magnetic elements are incorporated to enhance automatic closing and alignment. The outer cover 312 includes a first magnetic element 340, the inner tray 350 includes a second magnetic element 342, and the inner cover 316 includes a clearance opening 320 through which the first magnetic element 340 engages the second magnetic element 342. These magnetic components automatically attract and close the box when the cover is brought near the box body 348. Furthermore, a third magnetic element 344 on the outer cover 312 and a fourth magnetic element 346 on the inner cover 316 can engage to achieve secure closure of the inner cover 316 itself. A magnetic groove 322 in the inner cover 316 accommodates the fourth magnetic element 346, providing structural support and maintaining magnetic alignment. The inner tray 350 further includes a second handle 356 adjacent to the receiving slot 352, allowing the user to easily grasp and remove the light therapy device 100. The charging assembly 362 disposed between the inner tray 350 and the bottom shell 358 includes a circuit board 110, a first battery 366, and protection circuits (e.g., overcurrent, overvoltage, and short circuit protection). The connection terminal 368 enables efficient power delivery from the first battery 366 to the light therapy device 100. The inner tray 350 and the bottom shell 358 may be snap-fitted to facilitate maintenance access to the charging assembly 362.

In an embodiment, similar wireless communication and digital interaction modules may also be integrated into the light therapy patch and/or the charging case. The light therapy patch may include an NFC tag, Bluetooth® transceiver, or Wi-Fi module configured to pair with a user's mobile device or companion application for data synchronization, treatment tracking, or firmware updates. The communication module may also allow the user to view treatment history, battery level, or operating mode directly through a mobile interface.

The charging case may likewise include one or more of the digital interaction modules, such as an NFC tag, Bluetooth beacon, or QR code, enabling automatic pairing, device registration, or access to instructional content when the user scans or connects their mobile device. Such integration allows interactive communication between the charging case, the light therapy patch, and a user application or remote server, enhancing usability, brand engagement, and intelligent device management within the overall light therapy system.

In an embodiment, the application also provides a light therapy system comprising the light therapy device 100 and the above-described charging box. The light therapy device 100 includes a light-transmitting layer 300, a power supply assembly, and a backlight layer 306, with the power supply assembly located between the light-transmitting and backlight layers. The power supply assembly includes a light panel 302 and a second battery 304, wherein the light panel 302 is electrically connected to the charging assembly 362 via the connection terminal 368, enabling charging of the second battery 304. The backlight layer 306 includes a second charging port 308, which allows the light panel 302 and charging assembly 362 to interface electrically. A second electrode embedded in the second charging port 308 magnetically attracts and aligns with the first charging port 354 carrying the connection terminal a, ensuring automatic alignment and reliable conductivity during charging. Furthermore, the patch 100A may incorporate a memory alloy sheet 310 attached to the light-transmitting layer 300 and/or the backlight layer 306, enabling shape adaptation to the user's facial curvature for enhanced comfort and fit. Additionally, a Hall sensor or NFC module may be included for data detection, enabling intelligent identification, usage tracking, or automated charging activation. The combination of structural precision, magnetic alignment, and smart sensing features results in a compact, multifunctional, and user-friendly light therapy device that ensures both reliable operation and improved therapeutic performance.

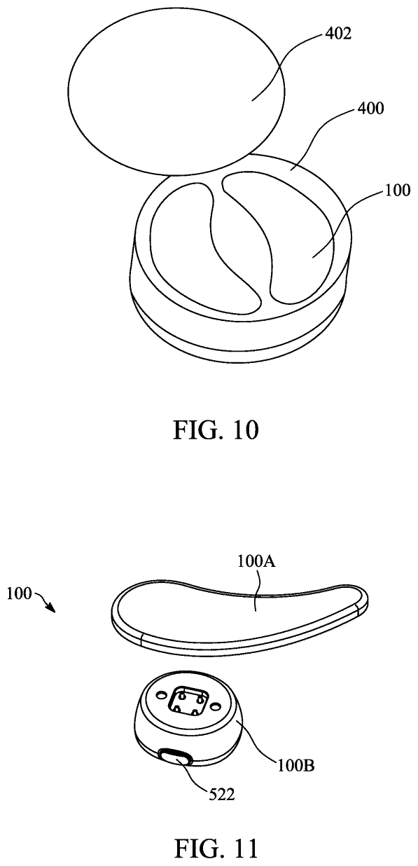

Referring to FIG. 10, in an embodiment, an alternate configuration of a storage box to store a light therapy device is disclosed, which provides a light therapy kit comprising a light therapy device 100 and a charging box 400. The charging box 400 includes a box body, an upper cover 402, and a lower cover, wherein the upper cover 402 and lower cover are disposed on two opposite sides of the box body along a first direction (X direction in FIG. 1). Both the upper cover 402 and the lower cover are rotatably connected to the box body about respective rotation axes extending along the first direction. The box body defines an accommodating cavity for receiving the light therapy device 100, which can be covered or opened by the rotation of the upper cover 402 relative to the box body. A storage cavity is formed between the lower cover and the box body, and the lower cover rotates to cover or open this storage cavity. The linkage structure between the box body, upper cover 402, and lower cover, combined with the rotation axes aligned along the first direction, enables the two covers to open and close in a parallel manner, ensuring smooth and stable motion. This design significantly reduces the space required in the vertical direction during operation compared to traditional flip-type boxes, allowing horizontal opening and one-handed operation even in compact environments. The box body further includes a charging mainboard and a power supply electrically connected, enabling the light therapy device 100 to be charged while stored in the accommodating cavity. Additionally, the inclusion of the lower cover forms a dedicated storage cavity for accessories such as spare patches, improving portability and convenience.

In an embodiment, the upper and lower covers are equipped with limiting mechanisms to enhance stability and durability. The box body includes a first limiting groove and a second limiting groove, both arc-shaped, while the upper cover 402 and the lower cover respectively include first and second limiting posts that slide within the corresponding grooves during rotation. These mechanisms effectively regulate the rotation range and prevent structural damage from excessive opening, while also minimizing shaking and improving user experience. The close engagement between each limiting post and groove further provides a partial locking function to prevent accidental opening during transport. A Hall sensor is arranged in the box body, aligned with a first magnet mounted in the upper cover 402. When the upper cover 402 is closed, the magnetic field detected by the Hall sensor signals the control system to automatically start charging the light therapy device 100, thereby realizing intelligent and automatic charging. When opened, the charging process stops immediately to prevent energy waste and overcharging. Additionally, a second magnet is arranged in the accommodating cavity of the box body, which magnetically cooperates with a third magnet in the light therapy device 100. This magnetic coupling ensures stable positioning, reduces wear associated with mechanical locks, and improves conductive connection reliability between the devices.

In an embodiment, the light therapy device 100 can be a patch-type device including a second shell, a second rechargeable battery, and a second phototherapy lamp arranged within the shell. The second shell has a flat structure comprising an opaque outer layer and a translucent inner layer, with a phototherapy circuit board between them to support efficient and comfortable light emission. In another embodiment, the light therapy device 100 includes a light therapy patch and a controller, wherein the controller, containing a first rechargeable battery, is detachably and magnetically connected to the patch, which includes a first shell and a first phototherapy lamp. The accommodating cavity of the box body includes a first cavity for the light therapy patch and a second cavity for the controller, ensuring compact and organized storage. The second cavity features a first opening recessed toward a second opening matching the controller's shape, facilitating both fixation and easy removal. Optionally, the box body may include two first cavities for storing two light therapy patches and a controller, allowing continued treatment by switching controllers. A hand-grip groove is formed between the two accommodating cavities to facilitate handling, while a non-slip pad is provided at the bottom of the lower cover to ensure stability and prevent surface damage during placement. The integrated rotation-based dual-cover structure of the charging box 400, in combination with the magnetic fixation and automatic charging features, provides a compact, intelligent, and user-friendly light therapy kit that enhances safety, convenience, and portability.

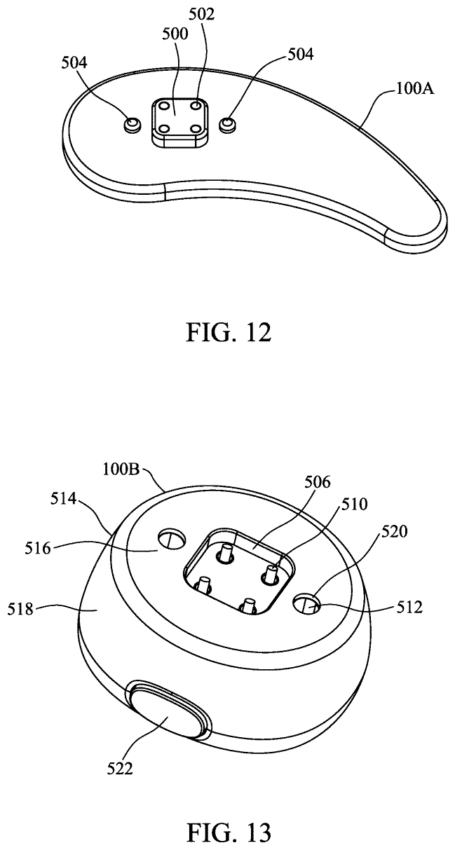

Referring to FIGS. 11, 12, and 13, the present invention provides a light therapy patch and a light therapy patch assembly. The light therapy patch includes a patch 100A and a detachable controller 100B, which are detachably connected. The patch 100A is configured to perform light therapy on the skin, and the detachable controller 100B supplies power to the patch 100A. The light-emitting side of the patch 100A is a flat surface intended to be attached to the skin surface, while the backlight side has a first raised structure 500, on which a first electrical connection portion 502 is provided. The detachable controller 100B has a first recessed structure 506 configured to receive the first raised structure 500, and the inner surface of the first recessed structure 506 has a second electrical connection portion 510. When the detachable controller 100B is mounted on the patch 100A, the first raised structure 500 extends into the first recessed structure 506, and the first electrical connection portion 502 and the second electrical connection portion 510 come into contact and conduct electricity. This structure precisely positions the detachable controller 100B and the patch 100A relative to each other, prevents sliding, and ensures a stable electrical connection even when the patch 100A is curved or subjected to facial movement. In some embodiments, at least one of the first electrical connection portions 502 and second electrical connection portions 510 is an elastic member, such as a conductive spring pin or conductive sheet, which allows compression during assembly and maintains electrical continuity during movement. Optionally, the electrical connection portions may be magnetic electrodes, forming both conductive and magnetic attraction forces, further stabilizing contact and preventing disconnection.

In certain embodiments, the first electrical connection portion 502 may be disposed on the sidewall or top wall of the first raised structure 500, while the second electrical connection portion 510 may be disposed on the sidewall or bottom wall of the first recessed structure 506. Multiple first electrical connection portions 502 and second electrical connection portions 510 may be provided in corresponding pairs, circumferentially distributed and evenly spaced around the centerline of the raised and recessed structures to ensure balanced and reliable conduction. For example, there may be four or three electrical connectors, with their centers forming a rectangle or circle, respectively, around the midline of the first raised structure 500. The patch 100A is provided with a first magnetic portion 504, and the detachable controller 100B is provided with a second magnetic portion 512, which are magnetically engaged. At least one of the first magnetic portion 504 and second magnetic portion 512 is a magnet, while the other may be made of iron or magnetic material. The magnetic attraction generated between the first magnetic portion 504 and the second magnetic portion 512 enhances the stability of the detachable connection between the patch 100A and the detachable controller 100B. Multiple first magnetic portions 504 may be arranged around the first raised structure 500, and multiple second magnetic portions 512 may be arranged around the first recessed structure 506 to prevent wobbling and improve alignment. Optionally, the first magnetic portion 504 may protrude from the patch surface, and the second magnetic portion 512 may be recessed into a positioning hole 520 on the detachable controller 100B, allowing the first magnetic portion 504 to extend into the positioning hole 520 for accurate positioning.

In an embodiment, the detachable controller 100B includes a control housing 514, a first battery, a key switch 522, and a control circuit board. The first battery, which may be a disposable dry cell, button cell, or rechargeable battery, and the control circuit board are both disposed within the control housing 514. The key switch 522, which may be mechanical or capacitive, is electrically connected with the control circuit board, the battery, and the second electrical connection portion 510 to form a power supply circuit. The key switch 522 enables the user to control the patch on/off state, brightness, and wavelength, providing flexible use. The control housing 514 has a first side 516, a second side, and a peripheral side 518 connecting them. The first recessed structure 506 is located on the first side 516, while the key switch 522 is arranged on the peripheral side 518. The direction from the first to the second side is vertical, and pressing the key switch 522 applies force in a lateral direction, which does not affect the vertical engagement between the patch 100A and the detachable controller 100B, ensuring consistent electrical contact. In traditional light therapy controllers, the control switch is generally positioned on the top surface, and when the user applies the patch to an acne-affected or inflamed skin area, pressing the top button can exert direct pressure on the skin, potentially causing discomfort, irritation, or bursting of acne. To overcome this limitation, the key switch 522 is positioned on the side of the controller (i.e., on the peripheral side 518), allowing for lateral switching operation without applying pressure to the treated skin surface. This arrangement ensures safer and more comfortable operation when treating sensitive or acne-prone skin areas, thereby improving therapeutic usability and user experience. In alternative embodiments, the key switch 522 may be disposed on the second side of the control housing 514, and the pressing direction may coincide with the installation direction of the detachable controller 100B. The patch 100A includes a surface-mount circuit board and a light-emitting portion that emits light of predetermined wavelengths to promote skin repair, remove acne, and enhance skin brightness.

In an embodiment, a light therapy patch assembly, which includes the light therapy patch described above and a charging box. The charging box includes a first accommodating cavity for receiving the patch 100A and a second accommodating cavity for receiving the detachable controller 100B. The bottom of the first accommodating cavity is provided with a second recessed structure matching the first raised structure 500, and the bottom of the second accommodating cavity is provided with a second raised structure matching the first recessed structure 506. This configuration allows the patch 100A and detachable controller 100B to be securely positioned within the charging box, preventing shaking or misalignment during transportation. In some embodiments, the patch 100A is magnetically positioned within the first accommodating cavity, and/or the detachable controller 100B is magnetically positioned within the second accommodating cavity. The patch 100A has the first magnetic portion 504, the detachable controller 100B has the second magnetic portion 512, the first accommodating cavity has a third magnetic portion, and the second accommodating cavity has a fourth magnetic portion. When the patch 100A is placed in the charging box, the first and third magnetic portions attract each other; when the detachable controller 100B is placed in the charging box, the second and fourth magnetic portions attract each other. Multiple third and fourth magnetic portions may be circumferentially arranged around the second recessed structure and the second raised structure, respectively. This magnetic positioning ensures that both the patch 100A and detachable controller 100B remain stable during storage and charging, while providing users with a compact, portable, and efficient light therapy assembly.

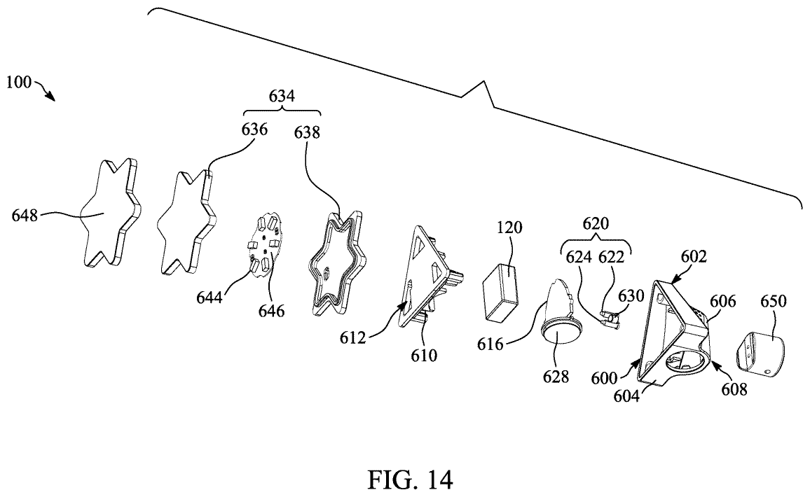

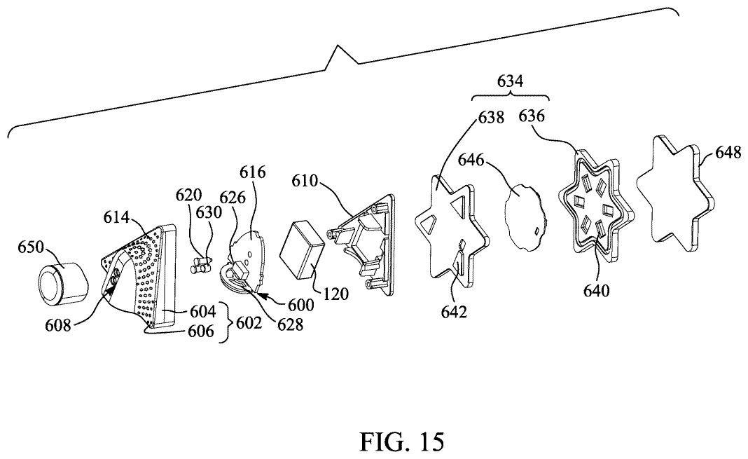

Referring to FIGS. 14 and 15, in an embodiment, an alternate configuration of a light therapy device is given. A light therapy device 100, which is used to irradiate the skin with light of specific wavelengths to improve skin condition, promote skin repair, and relieve inflammation. The light therapy device 100 includes a power supply box 600 and a patch 100A. The power supply box 600 comprises a box body, a first circuit board 616, and the rechargeable battery 120, wherein the box body is provided with a charging interface 620. The first circuit board 616 and the rechargeable battery 120 are housed within the box body, and the rechargeable battery 120 is electrically connected to the charging interface 620 through the first circuit board 616. The patch 100A, which includes a casing 634 and lamp beads 644 arranged therein, is fitted on the box body and electrically connected to the first circuit board 616. The box body, made of a hard material such as plastic, accommodates and protects the internal electronic components while providing pressure resistance. The rechargeable battery 120, being rechargeable, supplies power to the lamp beads 644 and can be recharged via the charging interface 620. In another implementation, a changeable coin battery may be provided instead of or in addition to the rechargeable battery 120, allowing users to conveniently replace the battery when depleted without requiring wired charging, thereby extending the device's portability and usability during travel or outdoor use. Further, in an embodiment, the charging head 650 may itself be configured as a removable battery module, capable of directly supplying power to the device when connected to the charging interface 620. The removable battery module can be detached and recharged separately, allowing quick battery replacement and continuous operation without waiting for recharging of the rechargeable battery 120. The patch 100A, typically made of flexible silicone, directly contacts the user's skin and delivers light of specific wavelengths, such as red, blue, or infrared, to achieve different therapeutic effects. The patch 100A is integrally arranged with the box body, allowing independent use even during charging.

In an embodiment, the box includes a connecting portion 604 and an extending portion 606. The connecting portion 604 connects to the casing 634, while the extending portion 606 extends outward from it, with the area of the connecting portion 604 being smaller than or equal to that of the casing 634 and its width greater than that of the extending portion 606. This ensures stability near the skin and ergonomic design for handling. The connecting portion 604 may be shaped as a square, triangle, or star to suit aesthetic or structural preferences. A switch 626 is provided on one end of the first circuit board 616 along the connecting portion 606, and a pressing member 628 is installed to operate it. The pressing member 628 transmits force horizontally (X-direction) to control the switch 626 without pressing the skin-facing patch, ensuring comfort. The switch 626 may also control lamp brightness or mode selection. The box body may also include a control panel for touch or button-based adjustments and a display. The charging interface 620 is located at the top end of the extending portion 606, on a flat area 608 designed to accommodate the charging head 650 conveniently. This arrangement ensures a safe distance from the patch 100A and allows comfortable charging.

In an embodiment, the box further comprises a box body 602 and a box cover 610 connected to form a storage cavity for the first circuit board 616 and the rechargeable battery 120. A first limiting structure 612 on the box cover 610 cooperates with a second limiting structure 642 on the casing 634 to improve alignment. The limiting structures may be protrusions or grooves, arranged correspondingly, and may include multiple sets for enhanced fitting accuracy. The patch 100A may have a hexagonal design, while the box cover 610 is triangular, forming an aesthetically coordinated structure. The box body 602 may further include a decorative pattern 614, such as a hexagonal groove on the extending portion 606, providing both anti-slip functionality and visual enhancement. Heat dissipation holes may also be provided on the box body to allow internal air exchange, preventing overheating and improving the reliability and lifespan of the electronic components. The lamp bead 644 is preferably an LED installed on a second circuit board 646 within the casing 634, connected to the rechargeable battery 120 via the first circuit board 616. The LEDs may emit red, blue, or infrared light, and can be combined as needed for various treatment purposes.

In an embodiment, the casing 634 includes a surface layer 636 and a bottom layer 638. The surface layer 636 is light-transmitting, made of transparent silicone, while the bottom layer 638 is opaque, made of or coated with light-blocking material such as metal or colored silicone. This design ensures that light from the lamp beads 644 efficiently reaches the skin without scattering backward. A reflective layer may be added inside the bottom layer 638 to direct more light toward the surface layer 636, enhancing therapeutic efficacy. The casing 634 is sealed and waterproof, with internal grooves 640 to house the lamp beads 644, reducing thickness. The light therapy device 100 may further include a gel patch 648 attached to the outer side of the patch 100A. The gel patch 648, which may contain moisturizing or medicinal ingredients, can be replaced by the user and enhances drug absorption under light exposure, improving comfort and efficacy. The charging interface 620 includes first electrode 622 and second electrode 624, made of conductive materials such as copper or gold-plated alloy. These electrodes may be magnetic elements or be accompanied by one or more magnetic components 630, such as NdFeB magnets, to enable magnetic charging with automatic alignment and stable electrical contact with the charging head 650. In some embodiments, the charging interface 620 may use spring pins or be designed as USB or Type-C ports. A charging cable with a detachable charging head 650 connects externally for recharging the rechargeable battery 120. This comprehensive design enables both independent operation and direct charging use, enhancing portability, reliability, and user comfort while improving overall therapeutic performance.

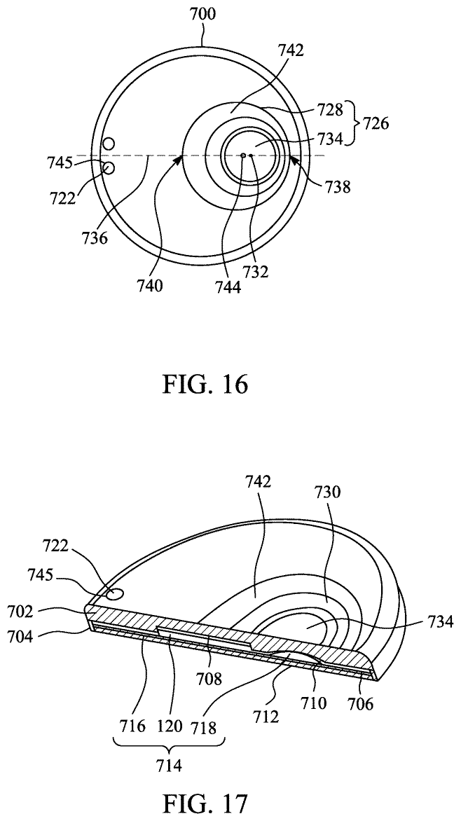

According to an embodiment of the present invention, referring to FIGS. 16 and 17, an alternate configuration of the light therapy device is provided. The light therapy device mainly comprises a base 700, a light-transmitting sheet 712, and a phototherapy component 714. A tactile structure 726 is provided on one side of the base 700, including a concave portion and/or a convex portion, arranged in an annular manner around a trigger portion 718 on the light board 716, which is positioned between the base 700 and the light-transmitting sheet 712. The tactile structure 726 may form either a closed or non-closed annular structure. This design allows the user to locate the trigger portion 718 by touch, enabling accurate blind operation and helping the user quickly identify the correct side of the device for fitting. The light-transmitting sheet 712, connected to the side of the base 700 opposite the tactile structure 726, protects the light board 716 while ensuring even light transmission to the skin surface. The phototherapy component 714 includes the light board 716, which carries the light source, such as LEDs, and the trigger portion 718, which controls functions such as switching the light board on or off. When the trigger portion 718 is pressed through the tactile portion 734, the light source emits therapeutic light of specific wavelengths for skin treatment. The tactile structure 726 further includes a corrugated structure 728 comprising alternately arranged concave and convex portions forming ripples, and a tactile portion 734 disposed within the innermost circle of ripples 730. The tactile portion 734 corresponds to the trigger portion 718 and is concentric with the innermost ripple 730, serving as the tactile focal point. This layered ripple arrangement provides tactile navigation, allowing users to accurately locate the trigger portion 718 even without visual assistance.

In an embodiment, the tactile portion 734 is arched outward and elastic, allowing it to deform when pressed and make contact with the trigger portion 718. The elasticity provides a physical feedback or “click,” confirming activation of the trigger portion 718 and enhancing user satisfaction. Each ripple 730 formed on the base 700 is concentrically arranged such that the geometric center 732 of each corrugation lies on the central axis 736. The outermost corrugation is designated as the first corrugation 742, which extends between a first side 738 and a second side 740 along the central axis 736. The geometric center 732 of the first corrugation 742 is defined as the first center 744. The geometric centers 732 of the remaining corrugations located within the first corrugation 742 are positioned between the first center 744 and the first side 738, thereby maintaining a symmetrical and structured corrugation pattern about the central axis 736. The height of the ripple 730 gradually decreases, and the width W gradually increases from the first side 738 to the second side 740, forming a tactile gradient that helps the user sense direction and locate the tactile portion 734 more easily. The base 700 defines a central axis 736, represented as an imaginary line passing through its geometric center 732. This gradient design not only enhances tactile feedback but also improves visual layering and aesthetic appeal. The base 700 is further provided with a through-hole 745, through which a charging electrode 722 connected to the rechargeable battery 120 on the light board 716 passes. The rechargeable battery 120, preferably a lithium-ion battery, is integrated within the light board 716 for compactness and efficient space use. The charging electrode 722 may be magnetic, allowing magnetic attraction with an external charger for automatic alignment and contact, thereby simplifying the charging process, reducing wear, and extending service life.

In an embodiment, the base 700 includes a cover body 702 and an annular wall body 704 vertically connected to its edge, together forming an accommodating cavity 706 for housing the light board 716. The cover body 702 is provided with the tactile structure 726 and the through-hole 745, while the light-transmitting sheet 712 covers the open side of the accommodating cavity 706 to seal the structure. A first limiting groove 708 and a second limiting groove 710, connected to the accommodating cavity 706, are formed on the inner side of the cover body 702 to restrict the positions of the rechargeable battery 120 and the trigger portion 718, respectively. This ensures precise alignment between the tactile structure 726 and trigger portion 718 and prevents displacement of internal components during use, thereby improving stability and durability. On the side of the light board 716 facing the light-transmitting sheet 712, at least one lamp bead is provided, including a red light bead and/or a blue light bead. The red light bead promotes blood circulation and collagen production for skin rejuvenation, while the blue light bead provides antibacterial and anti-inflammatory benefits, particularly effective for acne treatment. The trigger portion 718 may be a pot piece, producing a distinct click when pressed for clear tactile feedback. The light therapy device may be formed as a flexible device, wherein the base 700 is made of materials such as TPU, silicone, or TPE; the light-transmitting sheet 712 of PET or silicone; and the light board 716 of a flexible PCB (FPCB) using polyimide or similar substrates. This flexible configuration allows the device to closely conform to facial contours, ensuring uniform light distribution, high comfort, and adaptability to facial movements, thereby enhancing therapeutic efficiency and long-term wearability.

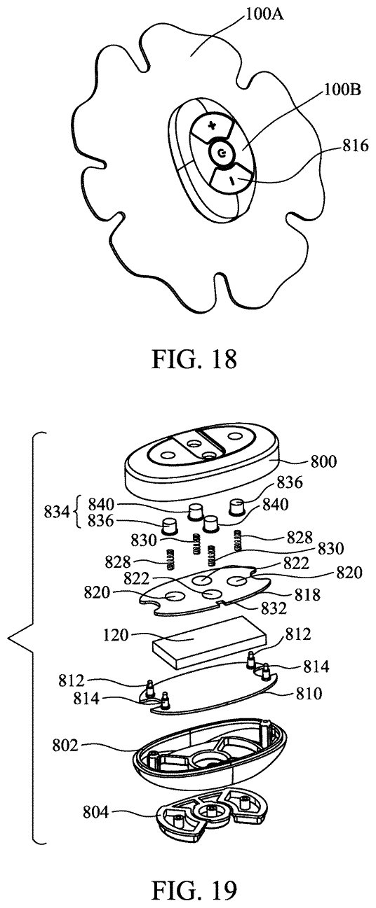

Referring to FIGS. 18 and 19, in an embodiment, a detachable controller 100B configured to be electrically connected to a patch 100A for controlling its operation is provided. The detachable controller 100B includes a housing, the rechargeable battery 120, the control board 810, the adapter board 818, and a terminal block 834, wherein the rechargeable battery 120, the control board 810, and the adapter board 818 are mounted within the housing. The control board 810 is used to control the patch 100A and is electrically connected to the rechargeable battery 120, while the adapter board 818 is electrically connected to the control board 810 and arranged on the opposite side of the rechargeable battery 120 along the thickness direction. The terminal block 834, located on the side of the adapter board 818 away from the control board 810, is electrically connected to the adapter board 818 and serves as an interface to an external circuit. The terminal block 834 may include two or more magnetic electrodes, conductive sheets, electrode caps, or electrode posts that enable the detachable controller 100B to connect to either the patch 100A or a charging station. When connected to the patch 100A, the terminal block 834 electrically mates with the magnetic terminals of the patch 100A to complete the circuit for control and operation. When connected to the charging station, it establishes electrical contact with the station's output terminals for charging the detachable controller 100B. The terminal block 834 extends partially outside the housing for connection while being secured internally by injection molding or adhesive sealing. The configuration wherein the control board 810 and adapter board 818 are placed on opposite sides of the rechargeable battery 120 facilitates a compact internal layout, reduces the cross-sectional area of the housing, and enables independent assembly of the terminal block 834 and rechargeable battery 120. The adapter board 818, being separate from the control board 810, allows sequential assembly and welding of the rechargeable battery 120 and terminal block 834, simplifying production. Optionally, the terminal block 834 and adapter board 818, as well as the control board 810 and adapter board 818, can be removably connected by abutting, snapping, or bolting.

In an embodiment, the terminal block 834 includes two first electrodes 836 and two second electrodes 840, wherein the first electrodes 836 are used for discharging (connection to the patch 100A) and the second electrodes 840 are used for charging (connection to the charging station). These electrodes are spaced apart to enhance safety and minimize interference between the charging and discharging circuits. A positioning slot is defined on the outer side of the housing near the terminal block 834, accommodating the two second electrodes 840 within the slot and the two first electrodes 836 on opposite sides. This design ensures proper alignment with a corresponding charging dock or the positioning platform of the patch 100A. The slot may be open at one end to improve the stability of electrical connections and provide a foolproof assembly feature. Within the housing, the first conductive plate 820 and second conductive plate 822 are disposed on the side of the adapter board 818 near the terminal block 834, corresponding to the first electrode 836 and second electrode 840, respectively. The first elastic members 828 and the second elastic members 830 (preferably springs) are positioned to maintain electrical contact between the first electrodes 836 and second electrodes 840, and the first conductive plate 820 and second conductive plate 822. Each spring may be seated in a positioning hole formed at the end of its respective electrode, or optionally welded to reduce resistance. The housing further includes first retaining rings and second retaining rings to secure the first electrodes 836 and second electrodes 840 through first and second openings, respectively. Additionally, first isolation rings and second isolation rings are arranged around each electrode to prevent short circuits and stabilize spring positioning. The adapter board 818 includes one or more conductive plates, which respectively abut with the third elastic member 812 and the fourth elastic member 814 on the control board 810 to enable a removable electrical connection between the control board 810 and the adapter board 818. Two pairs of third elastic members 812 and fourth elastic members 814 may be symmetrically arranged around the rechargeable battery 120 to balance pressure and ensure stable contact. The first electrode 836 and second electrode 840 may be magnetic members or magnets themselves, allowing magnetic engagement with the magnetic terminals of the patch 100A and the charging station, respectively. A retaining groove 832 on the adapter board 818 mates with a retaining bar on the housing, preventing reverse installation. A button 816 is provided on the control board 810 opposite a button cap 804 mounted on the housing, enabling user input. The device may comprise multiple buttons 816 for controlling different functions in the patch.

The housing includes a body 800 and a cover 802, wherein the adapter board 818 and terminal block 834 are installed in the body 800, and the control board 810 and the button cap 804 are attached to the cover 802. The body 800 and cover 802 can be secured by welding, adhesive bonding, or screws. The invention further provides a physical therapy device comprising the detachable controller 100B and a patch 100A, wherein the terminal block 834 of the detachable controller 100B is electrically connected to the patch 100A. This configuration simplifies assembly of the rechargeable battery 120, enhances structural integrity, reduces the internal space requirement, and improves space utilization within the detachable controller 100B. The patch 100A may be a light therapy patch or a heat therapy patch, allowing diverse treatment functions. The detachable controller 100B and patch 100A are removably connected, enabling convenient charging, replacement, and storage. The patch 100A includes magnetic terminals corresponding to the first electrodes 836, and a boss that supports these terminals to improve connection strength. The boss may include the positioning platform that aligns with the retaining groove 832 for precise positioning between the patch 100A and detachable controller 100B. A clearance hole may be formed on the positioning platform at a position corresponding to the second electrode 840, ensuring accurate alignment and stable contact. Overall, the described configuration of the detachable controller 100B and patch 100A achieves reliable electrical connectivity, improved assembly convenience, structural compactness, and versatile therapeutic applicability, all while enhancing the stability, safety, and usability of the physical therapy system.

In an embodiment, the patch 100A further comprises an ambient light sensor configured to control the switching ON/OFF of the therapeutic LED lamp beads based on whether the patch is secured on the user's skin. The patch 100A is provided with a secondary light source, such as a low-intensity visible light emitter.

When the patch 100A is powered on, the secondary light source is activated and emits visible light. The ambient light sensor is adapted to detect the presence or absence of surrounding light in the vicinity of the light therapy patch. When the patch 100A is attached to the user's skin, the emitted visible light from the secondary light source is blocked by the skin surface, resulting in a dark condition beneath the patch. Upon detecting the absence of light, the ambient light sensor transmits a signal to the control unit, which in turn activates the main LED lamp beads, such as red and blue LEDs, to emit therapeutic light towards the user's skin.

Moreover, when the patch 100A is not attached to the user's skin and is in a detached or idle state, such as when lying in a dark room or stored inside a packaging container, pressing the power switch causes the secondary light source to emit visible light. In this detached configuration, there is no obstruction to the emitted light, allowing the ambient light sensor to detect illumination in the surrounding environment. Upon detecting such light, the control unit refrains from activating the therapeutic LED lamp beads and protects the user's eyes from potential light exposure or damage caused by direct viewing of the therapy LEDs.

This intelligent control of the LED activation not only enhances energy efficiency but also ensures user safety by preventing inadvertent emission of high-intensity therapeutic light when the patch is not in contact with the skin. Such a control mechanism effectively protects the user's eyes from potential light exposure or damage caused by direct viewing of the therapy LEDs when the device is operated outside its intended skin-contact configuration.

Various modifications to these embodiments are apparent to those skilled in the art from the description and the accompanying drawings. The principles associated with the various embodiments described herein may be applied to other embodiments. Therefore, the description is not intended to be limited to the embodiments shown along with the accompanying drawings but is to provide the broadest scope consistent with the principles and the novel and inventive features disclosed or suggested herein. Accordingly, the invention is anticipated to hold on to all other such alternatives, modifications, and variations that fall within the scope of the present invention and appended claims.

1. A light therapy device comprising:
a light therapy patch having a flexible main body having an inner surface configured to adhere to skin of a user;
a control circuit disposed within the main body, the control circuit comprising a controller, and a plurality of light-emitting diodes configured to emit therapeutic light in at least one wavelength range; and
a pair of detachable microcurrent electrodes coupled to the control circuit and configured to deliver a microcurrent through the skin of the user.

2. The light therapy device of claim 1, wherein the plurality of light-emitting diodes is configured to emit therapeutic light in at least two wavelength ranges.

3. The light therapy device of claim 2, wherein the at least two wavelength ranges, including a red light wavelength between about 580 nm and 680 nm and an infrared wavelength between about 830 nm and 1080 nm.

4. The light therapy device of claim 1, wherein each of the detachable microcurrent electrodes comprises a conductive member disposed in a groove formed on the inner surface of the main body at both lateral ends.

5. The light therapy device of claim 1, wherein the control circuit and each of the detachable microcurrent electrodes are sealed within a waterproof housing having a pressing portion operatively coupled to a button switch configured to control a therapy mode of operation.

6. The light therapy device of claim 1, wherein the light therapy device further comprises an ambient light sensor configured to power on/off the light therapy device when the light therapy device is fixed to skin of the user.

7. The light therapy device of claim 1, further comprising a magnetic charging interface electrically connected to a power source and configured to receive electrical power from an external charging case.

8. The light therapy device of claim 7, wherein the external charging case comprising:
a box body defining an accommodating cavity for the light therapy device;
an upper cover and a lower cover rotatably connected to the box body along a rotation axis, a charging mainboard disposed in the box body, electrically connected to a pair of charging terminals positioned within the accommodating cavity; and
wherein the pair of charging terminals is magnetically alignable with the magnetic charging interface of the light therapy device to automatically initiate charging upon closure of the upper cover and the lower cover.

9. The light therapy device of claim 8, wherein the external charging case comprises a Hall sensor to detect the closure of the upper cover and the lower cover to automatically initiate charging of the light therapy device.

10. The light therapy device of claim 8, wherein the upper cover is rotatable on a horizontal axis of the main body.

11. A light therapy system comprising:
a light therapy device having a pair of primary electrodes, the light therapy device comprising a flexible casing having a plurality of light-emitting diodes configured to emit therapeutic light toward a user's skin; and
a controller configured to detachably attach to the light therapy device, the controller comprising:
a housing defining an internal cavity;
a control board disposed within the internal cavity and configured to control the operation of the light therapy device;
a battery disposed within the internal cavity and electrically connected to the control board;
an adapter board electrically connected to the control board and comprising at least one pair of secondary magnetic electrodes configured to magnetically and electrically engage with the primary electrodes of the light therapy device; and
a user interface electrically connected to the control board and configured to switch power and operation modes.

12. The light therapy system of claim 11, wherein the secondary magnetic electrodes include a pair of first electrodes for discharging power to the light therapy device and a pair of second electrodes for receiving charging current, the pair of first electrodes and the pair of second electrodes are spatially separated to reduce electrical interference.

13. The light therapy system of claim 11, wherein the light therapy device has a first interface portion and the controller has a second interface portion, the first interface portion is configured to mechanically and electrically connect to the second interface portion.

14. The light therapy system of claim 13, wherein the first interface portion and the second interface portion comprise one or more magnetic alignment elements and electrical contacts configured to provide a secure attachment and electrical communication between the controller and the light therapy device.

15. The light therapy system of claim 13, wherein the first interface portion comprises a protruded structure and the second interface portion comprises a recessed structure configured to mate with the protruded structure of the first interface portion.

16. The light therapy system of claim 11, wherein the user interface includes a multi-function button configured to switch between a phototherapy mode, a microcurrent mode, and a combined therapy mode.

17. The light therapy system of claim 16, wherein the multi-function button is a side-mounted switch positioned on a peripheral side of the controller to prevent direct pressure on a skin-contacting surface of the light therapy device.

18. The light therapy system of claim 11, wherein the adapter board includes conductive plates abutting spring-type elastic members on the control board to maintain detachable electrical control.

19. The light therapy system of claim 11, further comprising a charging case configured to receive and charge the light therapy device and the controller through respective magnetically aligned charging electrodes.

20. The light therapy system of claim 11, wherein the controller comprises a removable battery module or coin cell battery configured for replacement.
